(19) United States
(12) Patent Application Publication
(10) Pub. No.: US 2007/0141149 A1
(43) Pub. Date: Jun. 21, 2007

(54) Controlled-release pharmaceutical formulation

(76) Inventors: Polonca Kuhar, Ljubljana (SI); Judita Sirca, Ljubljana (SI)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/583,440

(22) PCT Filed: Dec. 22, 2004
(86) PCT No.: PCT/SI04/00044

§ 371 Date: Jun. 16, 2006

(30) Foreign Application Priority Data
Dec. 23, 2003 (SI) P-200300317

(51) Int. Cl. A61K 9/24; A61K 31/18

(52) U.S. Cl. 424/471; 514/602

(57) ABSTRACT

In the present invention, a new pharmaceutical formulation with controlled release of the freely water soluble low-dose active substance used for at the most once daily administration is disclosed. The active substance is maintained at a suitable therapeutic concentration in the blood throughout at least a 24 hour period independent of the physiological pH value to which the pharmaceutical formulation is exposed.

### FIELD OF THE INVENTION

The present invention belongs to the field of pharmaceutical technology and relates to a controlled-release pharmaceutical formulation used for at the most once daily dosing.

More particularly, the invention relates to a pharmaceutical formulation of controlled release pellets comprising in the core a low dose of an active substance which is freely soluble in water and optional coating. Release of the active substance from the core is controlled and independent of the physiologic pH value of the environment in which the core is placed.

### BACKGROUND OF THE INVENTION

There is a constant need for safe and at the same time conveniently administrable pharmaceutical formulations with controlled release of the active substance which are suitable for at the most once daily administration.

When an active substance is administered in very low doses, it may be freely soluble in water and therefore rapidly absorbed, so that achieving sustained release of such active substance is of key importance for maintaining therapeutic plasma concentrations.

Controlled-release pharmaceutical formulations with pH-dependent systems are known in the state of the art. In such systems, irrespective of the pharmaceutical formulation, variability in plasma concentrations of the active substance among individuals is great due to inter-individual differences (such as different gastric emptying, changing of pH values along the gastrointestinal tract, etc.).

A method for controlled release of a freely water-soluble active substance in low doses is described in the article by Cowen J. A., Griffin A., Hayward M. A. and Grattan T. J.; 15th Pharmaceutical Technology Conference, Oxford UK, 1996. Pellets were prepared by applying first the active substance (10% by weight) to neutral sugar-starch cores and then applying a coating having the function of release control.

An example of an active substance with the described characteristics is tamsulosin which is typically dosed in extremely low concentrations (e.g. about 0.2% by weight of a formulation). Tamsulosin is a selective antagonist of α_{1A} and α_{1D} adrenergic receptors. It is indicated for the treatment of benign prostatic hyperplasia. By selective and competitive binding to α₁ postsynaptic receptors, it relaxes smooth muscles in the prostate and the urinary bladder neck thereby increasing the urinary flow, facilitating urination and improving other symptoms of benign prostatic hyperplasia.

Orally administered tamsulosin on an empty stomach has almost 100% bioavailability. When taken during meals, its bioavailability as well as cₘₐₓ are decreased.

Tamsulosin from immediate release formulations is rapidly absorbed and plasma concentrations increase quickly. By developing modified release pharmaceutical formulations, an important step in improving the tolerance and prolonging activity of the active substance can be made. With modified release formulations the likelihood of causing vasodilatation and related cardiovascular side effects is diminished.

However, after single- or multiple-dose administration of the commercially marketed controlled release formulation of tamsulosin, considerable inter-individual variability in plasma concentrations is observed (Lyseng-Williamson K. A., Jarvis B., Wagstaff A. J; Drugs, 2002, vol. 62, no. 1, pp. 135-167(33) Adis International).

Pharmaceutical formulations for controlled release of tamsulosin are disclosed in the following patent documents:

In U.S. Pat. No. 4,772,475 an uncoated granulation formulation for controlled release is disclosed whereby difficulties in applying gastroresistant coatings are mentioned.

Both WO 03/039530 and WO 03/039531 disclose dry compressed tablets comprising tamsulosin; in the latter application, matrix tablets having a modified release are disclosed.

In DE 202 19 293, pellets comprising tamsulosin are disclosed, in which the coating mass calculated on a dry pellet core basis is 2.5-15%, preferably 8-12%.

Pellets are prepared by granulation, drying, sieving to the size of 0.3-0.9 mm, coating and re-drying. Tamsulosin is released in a pH-dependent manner. It is reported therein that use of agents which would release the active substance in a manner independent of the pH environment would prevent release of the active substance after the contact of the pellet core coating with a body fluid. HMPC is cited as an example of such an agent.

Thus in patent and related literature from this field, no references can be found to solve the problem of providing a pharmaceutical formulation (particularly in pellet form) that would allow controlled release of tamsulosin and/or active substances having similar characteristics in a pH-independent manner

The present invention is aimed at preparing a pH-independent system for controlled release of very low doses of an active substance, such as tamsulosin, which is freely soluble in water, thereby maintaining an adequate therapeutic concentration of the active substance in blood throughout 24 hours enabling at the most once daily administration.

### SUMMARY OF THE INVENTION

In the first aspect, the invention concerns a controlled release pharmaceutical formulation comprising a pellet core from which a low dose active substance which is freely soluble in water can be released in a controlled manner independently from pH thereby providing a lower biological variability.

In another aspect, the invention concerns a controlled release pharmaceutical formulation comprising a pellet core comprising at least one insoluble permeable polymer and at least one surfactant and optionally other excipients.

In another aspect, the invention concerns a process for the preparation of such pharmaceutical formulations comprising preparation of the blend of the ingredients for the core, granulation, extrusion and spheronization, drying and optionally coating.

In another aspect, the invention concerns a use of such pharmaceutical formulations with tamsulosin or pharmaceutically acceptable salts thereof for the preparation of a medicament for the treatment of benign prostatic hyperplasia.

### DETAILED DESCRIPTION OF THE INVENTION

We have surprisingly found that by using different insoluble permeable polymers, pH-independent release of water-soluble drugs administered in low doses can be achieved.

By controlled release, maintaining therapeutic concentrations over at least 24 hours, optionally longer, thereby allowing once daily or less frequent dosing, is meant.

The active substance incorporated into the pellet core of the formulation of the present invention is generally administered in low doses, and being thus freely water soluble and rapidly absorbed into the body. An example of the active substance with these characteristics is tamsulosin or any pharmaceutically acceptable salts thereof. In the context of the present invention “low dose” means such a low concentration of the active substance to be freely water soluble.

In addition to the active substance, the pellet core of the formulation of the invention comprises microcrystalline cellulose, at least one insoluble permeable polymer and optionally surfactants and other excipients.

Microcrystalline cellulose may be of any commercially marketed form, as well as silicified microcrystalline cellulose and the like. The amount of microcrystalline cellulose in the pellet core can be from about 60 to about 95%, preferably about 75-90%, more preferably about 85%.

For release control, pellet cores can comprise different insoluble permeable polymers in the form of powders, granules or water dispersions which enable pH independent release of the active substance. We have surprisingly found that for this purpose, selected acrylic polymers are particularly suitable, such as polymers or copolymers of acrylic or methacrylic acid or esters of acrylic or methacrylic acid, optionally having functional groups, among them particularly copolymers of methacrylic esters with trimethylammonioethyl- or ammonioethyl- or similar functional groups, copolymers of methacrylic acid and methacrylic esters, copolymers of methacrylic esters, further different types of alkylcelluloses, such as e.g. ethylcellulose or methylcellulose or different combinations thereof. Particularly suitable is the water insoluble copolymer of ethylacrylate and methylmethacrylate in a ratio of 2:1, in the form of a 30% water suspension. The portion of such polymer in the pellet core is from about 7 to about 27%, preferably about 10-20%, more preferably about 14-15%.

Surfactants may be ionic or non-ionic. Suitable examples are sorbitan oleate, sorbitan laurate, sodium lauryl sulphate, polyoxyethylene sorbitan fatty acid esters, such as Polysorbate®, or a combination thereof. The percent of the surfactants is from about 0.10 to about 0.20%, preferably about 0.15%.

The diameter of pellet cores is usually from about 0.5 to about 2.00 mm, preferably from about 0.5 to about 1.25 mm.

A coating may be applied onto the core. Optionally such a coating comprises at least one polymer soluble at higher pH values, that is, higher than about pH 5.5, and at least one polymer which solubility is pH independent. Such a coating can ensure additional release control of the active substance thereby allowing less than 10% of the active substance to be released in the first two hours after ingestion.

A dispersion comprising about 15-20% of dry substance has been found to be preferable for coating.

In addition to polymers, the coating can also comprise talc. The weight ratio of polymer to talc is about 2:1. Demineralised water is used as a solvent.

The polymer soluble at higher pH values is selected from copolymers of methacrylic acid and acrylate and/or ethylacrylate or esters of hydroxyalkycelluloses.

The polymer having a pH independent solubility is selected from the same group as for the pellet core.

The amount of the applied coating can be from about 5 to about 25%, preferably about 5-10%, more preferably about 5-8%, most preferably about 7% by weight relative to the weight of dried pellet cores.

The pellet cores are prepared by processes conventional in pharmaceutical technology. For instance, a blend of tamsulosin, microcrystalline cellulose, surfactants, a release sustaining polymer and demineralised water can be mixed to homogeneity. The granulate can then be extruded, and the extrudate spheronized. The resulting cores can be dried in a fluid-bed drier.

The coating is applied preferably by spraying the dispersion in fluid-bed devices, such as e.g. a Wurster chamber, Huettlin Kugelcoater and the like. The coating parameters differ from device to device; the temperature of the product should be kept below 30° C. Pellets prepared in such a manner should then be spread out on trays to dry at about 40-60° C. for about 2 to about 24 hours.

Pellets can be filled into capsules of a suitable size or sachets or compressed into tablets.

The pharmaceutical formulation according to the present invention comprising tamsulosin or pharmaceutically acceptable salts thereof can be used for the treatment of benign prostatic hyperplasia or other diseases or disorders treatable with tamsulosin, either alone or in the combination with other active principles.

### EXAMPLES

The present invention is illustrated but in no way limited by the following examples:

### Example 1

| CORE (core weight = 200 mg) | | |
|---|---|---|
| Tamsulosin hydrochloride | 0.400 | mg |
| Microcrystalline cellulose | 146.200 | mg |
| Sodium lauryl sulphate | 0.300 | mg |
| Eudragit NE 30 D ® | 177.000 | mg |
| Demineralised water | 5.00 | mg |

Method of Preparation:

Tamsulosin hydrochloride and microcrystalline cellulose are combined and mixed. Sodium lauryl sulphate (Texapon K12®) is dissolved in water and the solution is added to the basic blend. A dispersion of Eudragit NE 30 D® and demineralised water is added and mixed. From the homogeneous blend, pellet cores are made using the method of extrusion and spheronization. The prepared cores may be coated with the coating as described in examples 4 and 5.

### Example 2

| CORE (core weight = 200 mg) | | |
|---|---|---|
| Tamsulosin hydrochloride | 0.400 | mg |
| Microcrystalline cellulose | 153.300 | mg |
| Polysorbate 80 V ® | 0.300 | mg |
| Eudragit NE 30 D ® | 153.333 | mg |
| Demineralised water | 45.000 | mg |

Method of Preparation:

Tamsulosin hydrochloride and microcrystalline cellulose are combined and mixed. Polysorbate 80® is dissolved in water and the solution is added to the basic blend. A dispersion of Eudragit NE 30 D® and demineralised water is added and mixed. From the homogeneous blend, pellet cores are made using the method of extrusion and spheronization. The prepared cores may be coated with the coating as described in examples 4 and 5.

### Example 3

| CORE (core weight = 200 mg) | | |
|---|---|---|
| Tamsulosin hydrochloride | 0.400 | mg |
| Microcrystalline cellulose | 159.300 | mg |
| Polysorbate 80 V ® | 0.300 | mg |
| Eudragit NE 30 D ® | 133.333 | mg |
| Demineralised water | 5.00 | mg |

Method of Preparation:

Tamsulosin hydrochloride and microcrystalline cellulose are combined and mixed. Polysorbate 80® is dissolved in water and the solution is added to the basic blend. A dispersion of Eudragit NE 30 D® and demineralised water is added and mixed. From the homogeneous blend, pellet cores are made using the method of extrusion and spheronization. The prepared cores may be coated with the coating as described in examples 4 and 5.

### Example 4

| COATING (17.6% application, | | |
|---|---|---|
| coating weight = 35.2 mg) | | |
| Eudragit NE 30 D ® | 58.520 | mg |
| Eudragit L 30 D-55 ® | 19.610 | mg |
| Talc | 11.760 | mg |
| Demineralised water | 86.280 | mg |

Total weight of coated pellets in one capsule = 235.3 mg

Method of Preparation:

Dry pellet cores are coated with the coating dispersion prepared in three steps. First, both polymers dispersions are diluted with demineralised water and mixed. A suspension of talc in demineralised water is prepared separately. Then the talc suspension is added to the diluted Eudragit L 30 D-55® dispersion and mixed Then the diluted Eudragit NE® dispersion is added and mixed again. The resulting dispersion is used for coating the pellet cores in a fluid-bed device.

### Example 5

| COATING (25% application, | | |
|---|---|---|
| coating weight = 50 mg) | | |
| Eudragit NE 30 D ® | 83.330 | mg |
| Eudragit L 30 D-55 ® | 27.780 | mg |
| Talc | 16.670 | mg |
| Demineralised water | 122.250 | mg |

Total weight of coated pellets in one capsule = 250 mg

Method of Preparation:

Dry pellet cores are coated with the coating dispersion prepared in three steps. First, both polymer dispersions are diluted with demineralised water and mixed. A suspension of talc in demineralised water is separately prepared. Then the talc suspension is added to the diluted Eudragit L 30 D-55® dispersion and mixed Then the iluted Eudragit NE® dispersion is added and mixed again. The resulting dispersion is used for coating the pellet cores in a fluid-bed device.

### Example 6

| CORE (core weight = 200 mg) | | |
|---|---|---|
| Tamsulosin hydrocloride | 0.400 | mg |
| Microcrystalline cellulose | 139.300 | mg |
| Polysorbate 80 V ® | 0.300 | mg |
| Ethylcellulose | 60.00 | mg |
| Demineralised water | 220.000 | mg |

| COATING (25% application, | | |
|---|---|---|
| coating weight = 50 mg) | | |
| Eudragit NE 30 D ® | 83.330 | mg |
| Eudragit L 30 D-55 ® | 27.780 | mg |
| Talc | 16.670 | mg |
| Demineralised water | 122.250 | mg |

Total weight of coated pellets in one capsule = 250 mg

Method of Preparation:

Tamsulosin hydrocloride and microcrystalline cellulose are combined and mixed. Ethylcellulose, aqueous Polysorbate® solution and demineralised water are added and mixed. From the homogeneous blend, pellet cores are made using the method of extrusion and spheronization.

Dry pellet cores are coated with the coating dispersion prepared in three steps. First, both polymer dispersions are diluted with demineralised water and mixed. A suspension of talc in demineralised water is separately prepared. Then the talc suspension is added to the diluted Eudragit L 30 D-55® dispersion and mixed Then diluted Eudragit NE® dispersion is added and mixed again. The resulting dispersion is used for coating the pellet cores in a fluid-bed device.

### Example 7

| CORE (core weight = 200 mg) | | |
|---|---|---|
| Tamsulosin hydrochloride | 0.400 | mg |
| Microcrystalline cellulose | 171.000 | mg |
| Sodium lauryl sulphate | 0.300 | mg |
| Eudragit NE 30 D | 93.330 | mg |
| Demineralised water | 80.000 | mg |

Method of Preparation:

Tamsulosin hydrochloride and microcrystalline cellulose are combined and mixed. Sodium lauryl sulphate (Texapon K12) is dissolved in water and the solution is added to the basic blend. Dispersion of Eudragit NE 30 D and demineralised water is added and mixed. From the homogeneous blend, pellet cores are made using the method of extrusion and spheronization. The prepared cores may be coated with the coating as described in examples 4 and 5.

The coating dispersion in all examples contains 20% of dry substance. The ratio of polymer weight to talc weight is 2:1, the ratio of polymers is 3:1 in favour of Eudragit NE 30D®.

Both polymers are in the form of a 30% aqueous dispersion.

1. A controlled release pharmaceutical formulation comprising a pellet core from which a low dose active substance freely soluble in water can be released in a controlled manner independently from pH thereby providing a lower biological variability.

2. A controlled release pharmaceutical formulation characterised in that it comprises a pellet core comprising at least one insoluble permeable polymer and at least one surfactant and optionally other excipients.

3. The pharmaceutical formulation according to claim 2 wherein said insoluble permeable polymer is selected from the group of acrylic polymers or alkylcelluloses or hydroxyalkylcelluloses or a combination thereof.

4. The pharmaceutical formulation according to claim 3 wherein said insoluble permeable polymer is a copolymer of ethylacrylate and methylmethacrylate in a ratio of 2:1, optionally being in the form of a 30% aqueous dispersion.

5. The pharmaceutical formulation according to claim 1 wherein the diameter of the pellet cores is from about 0.5 to about 1.25 mm.

6. The pharmaceutical formulation according to claim 1 wherein said pellet core is coated with a gastroresistant and/or release controlling coating.

7. The pharmaceutical formulation according to claim 6 wherein the mass of the applied coating is from about 5 to about 10% relative to the mass of dried pellet cores.

8. The pharmaceutical formulation according to claim 7 wherein the mass of the applied coating is from about 5 to about 8% relative to the mass of dried pellet cores.

9. The pharmaceutical formulation according to claim 6 wherein the coating comprises at least one polymer soluble at pH values higher than about 5.5 and at least one polymer with a pH independent solubility.

10. The pharmaceutical formulation according to claim 9 wherein said polymer soluble at higher pH values is an anionic copolymer of methacrylic acid and ethylacrylate and said polymer with pH independent solubility is a copolymer of ethylacrylate and methylmethacrylate.

11. The pharmaceutical formulation according to claim 1 wherein the pellets are filled into capsules or sachets or compressed into tablets.

12. The pharmaceutical formulation according to claim 1 wherein the pellet cores are prepared by using the methods of extrusion and spheronization.

13. The pharmaceutical formulation according to any of the preceding claims wherein the freely soluble low-dose active substance is tamsulosin or a pharmaceutically acceptable salt thereof.

14. A process for the preparation of pharmaceutical formulations according to claim 1 characterised in that it comprises the following steps: preparation of the blend of the ingredients for the core, granulation, extrusion and spheronization, drying and optionally coating.

15. Use of the pharmaceutical formulation according to claim 13 for the preparation of a medicament for the treatment of benign prostatic hyperplasia.
